(19) United States
(12) Patent Application Publication
(10) Pub. No.: US 2014/0039466 A1
(43) Pub. Date: Feb. 6, 2014

(54) TUBE

(71) Applicant: TERUMO KABUSHIKI KAISHA, Shibuya-ku (JP)

(72) Inventor: Kyouta Kimura, Ashigarakami-gun (JP)

(73) Assignee: TERUMO KABUSHIKI KAISHA, Shibuya-ku (JP)

(21) Appl. No.: 13/954,414

(22) Filed: Jul. 30, 2013

(30) Foreign Application Priority Data
Jul. 31, 2012 (JP) 2012-169853

(51) Int. Cl. A61M 1/04

(52) U.S. Cl. CPC A61M 1/04
USPC 604/540

(57) ABSTRACT

Disclosed herein is a tube mounted in a hole that makes a body surface and inside of lung parenchyma communicate with each other, the tube including: a main body portion that has a hole part having one end opened to the inside of the lung parenchyma and the other end opened to outside of the body surface; and a flow rate adjuster that is provided for the main body portion and adjusts a flow rate of air flowing in the hole part.

The present disclosure contains subject matter related to that disclosed in Japanese Priority Patent Application JP 2012-169853 filed in the Japan Patent Office on Jul. 31, 2012, the entire content of which is hereby incorporated by reference.

### BACKGROUND OF THE INVENTION

1. Field of the Invention

The present invention relates to tubes, and specifically relates to a tube for degassing mounted in a hole that makes a body surface and the inside of a lung communicate with each other.

2. Description of the Related Art

Heretofore, the chronic obstructive pulmonary disease (COPD), which is a progressive disease of the airways, is known. In a patient with this COPD, the alveolar walls of the lung tissue gradually weaken due to debilitation and the elastic recoil is lost. Accordingly, lung hyperinflation is caused and it becomes impossible to reduce the residual volume of the lungs. As this disease progresses, the inspiratory capacity and air exchange surface area of the lungs decrease, and finally breathing becomes difficult.

There has been proposed a treatment in which pneumostomas (lung holes) are created for a patient affected with the COPD and the residual air in the lungs is reduced from these pneumostomas (refer to e.g. JP-T-2011-512233 (Patent Document 1)).

In the treatment described in this Patent Document 1, on the front of the chest, the pneumostomas are formed at a site between the third and fourth ribs on the right lung and at a site on the fourth or fifth intercostal space under the left arm. These pneumostomas are surgically created by joining an artificial channel passing through the chest wall with an opening that passes through the visceral membrane of the lung and comes into parenchymal tissue of the lung. A tube having a filter is inserted into these pneumostomas and this tube is kept at a predetermined place by an external component secured to the skin of the patient.

By such pneumostoma and tube, an extra pathway for exit of exhaled air from the lung can be formed and the residual volume in the lung and the intrathoracic pressure can be reduced. That is, these pneumostoma and tube allow bypassing the natural airways impaired by COPD.

### SUMMARY OF THE INVENTION

However, the tube described in the above-mentioned Patent Document 1 has a problem that it is impossible to adjust the discharge amount of the residual air according to the residual functions of the lung, the individual difference in various kinds of lung functions, and the degree of progression of symptoms.

An object of the present invention is to provide a tube allowing adjustment of the discharge amount.

A tube according to one embodiment of the present invention is a tube mounted in a hole that makes a body surface and the inside of lung parenchyma communicate with each other. The tube includes a main body portion that has a hole part having one end opened to the inside of the lung parenchyma and the other end opened to the outside of the body surface, and a flow rate adjuster that is provided for the main body portion and adjusts the flow rate of air flowing in the hole part.

According to the above-described one embodiment, due to the provision of the flow rate adjuster that adjusts the flow rate of the air flowing in the hole part of the main body portion, the flow rate of the air that flows in this hole part and flows between the lung parenchyma and the outside of the body surface can be adjusted. Therefore, the discharge amount of the air discharged from the lung parenchyma to the outside of the body surface can be adjusted according to the residual functions of the lung, the individual difference in various kinds of lung functions, and the degree of progression of symptoms.

In the above-described one embodiment, it is preferable that the flow rate adjuster includes a diameter adjuster that is provided in the hole part and adjusts the diameter of a flow path of the air flowing in the hole part according to an applied pressure and a pressure adjuster that adjusts the pressure applied to the diameter adjuster.

According to the above-described one embodiment, the diameter adjuster of the flow rate adjuster adjusts the diameter of the flow path of the air flowing in the hole part according to the pressure adjusted by the pressure adjuster. According to this, decreasing the diameter of this flow path can decrease the flow rate of the air, and increasing the diameter of this flow path can increase the flow rate of the air. Therefore, the discharge amount of the air discharged from the lung parenchyma to the outside of the body surface can be surely adjusted.

In the above-described one embodiment, the following configuration is preferable. Specifically, the main body portion has a step part that is formed in the hole part and extends toward the radial outside of the hole part. The diameter adjuster is disposed on the step part and has an elastic member having an annular shape. The elastic member has a penetration hole penetrating the elastic member. The inner diameter of the penetration hole is adjusted according to the applied pressure. The pressure adjuster has a pressing part that advances and retracts in the hole part and applies a pressure to the elastic member.

According to the above-described one embodiment, the elastic member disposed on the step part in the hole part is pressed to be elastically deformed by the pressing part that advances and retracts in the hole part. Thereby, the inner diameter of the penetration hole of this elastic member is adjusted. This can easily adjust the inner diameter of the penetration hole according to the advancement/retraction amount of the pressing part. Therefore, the diameter of the flow path of the air discharged from the lung parenchyma to the outside of the body surface can be surely adjusted and hence the discharge amount of the air can be adjusted easily and surely.

In the above-described one embodiment, the following configuration is preferable. Specifically, the main body portion has a helical thread groove that is formed in one of an outer surface of the main body portion and an inner surface of the hole part and is along the center axis of the main body portion. The pressure adjuster has a thread screwed to the thread groove and is so configured as to be advanceable and retractable along the center axis of the main body portion.

According to the above-described one embodiment, because of the configuration in which the pressure adjuster advances and retracts relative to the main body portion based on the screwing of the thread groove and the thread, the pressure applied to the elastic member by the pressing part can be kept with the simple configuration. Therefore, the return of the inner diameter of the penetration hole adjusted through the elastic deformation of this elastic member to the original diameter can be suppressed and the adjusted discharge amount of the air can be surely kept. Furthermore, by rotating the pressure adjuster, this pressure adjuster and hence the pressing part can be made to advance and retract along the center axis of the main body portion. Thus, the pressure applied to the elastic member can be adjusted more easily and moreover the discharge amount of the above-described air can be adjusted more easily.

In the above-described one embodiment, it is preferable that the diameter adjuster is provided in the hole part and expands toward the radial inside of the hole part according to the pressure of a supplied medium to adjust the diameter of the flow path and the pressure adjuster adjusts the amount of medium supplied to the diameter adjuster.

As the medium, besides a gas such as air, a liquid such as a normal saline solution can be exemplified.

According to the above-described one embodiment, the diameter adjuster expands according to the amount of medium supplied by the pressure adjuster to adjust the diameter of the above-described flow path. Thus, the discharge amount of the above-described air can be surely adjusted. Furthermore, by adjusting the amount of injected medium, the diameter of the flow path can be easily adjusted and the adjustment of the discharge amount of the air can also be easily carried out.

In the above-described one embodiment, it is preferable that the pressure adjuster includes an injection path that is formed in the main body portion and injects the medium to the diameter adjuster and a check valve that is provided on the opposite side to the side of the diameter adjuster in the injection path and prevents reverse flow of the injected medium.

According to the above-described one embodiment, the check valve is provided for the injection path to inject the medium to the diameter adjuster. This can suppress the occurrence of the situation in which the injected medium flows in reverse to leak out to the external and the adjusted diameter of the flow path is changed. Therefore, the adjusted discharge amount of the air can be kept.

### BRIEF DESCRIPTION OF THE DRAWINGS

FIG. 1 is a sectional view showing a mounting example of a tube according to a first embodiment of the present invention;

FIG. 2 is a sectional view showing the tube in the first embodiment;

FIG. 3 is a sectional view showing the tube in the first embodiment;

FIG. 4 is a sectional view showing a tube according to a second embodiment of the present invention;

FIG. 5 is a sectional view showing a mounting example of a tube according to a third embodiment of the present invention; and

FIG. 6 is a sectional view showing the tube in the third embodiment.

### DETAILED DESCRIPTION OF THE PREFERRED EMBODIMENTS

### First Embodiment

A first embodiment of the present invention will be described below based on the drawings.

### [Configuration of Tube]

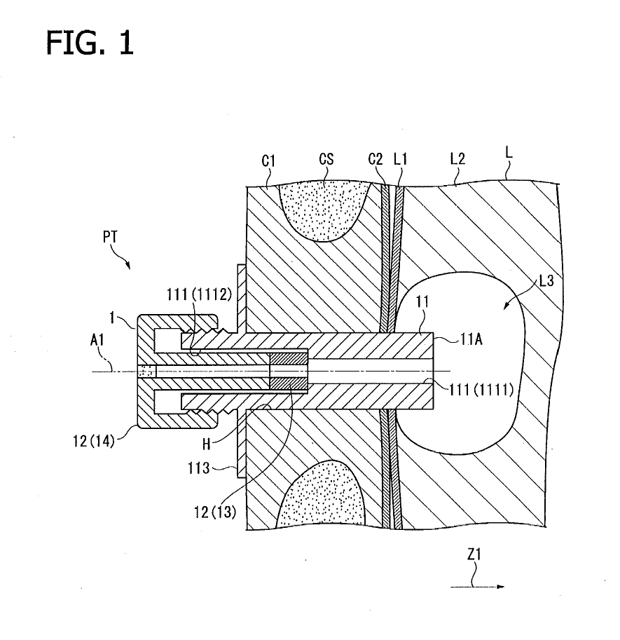

FIG. 1 is a sectional view showing a mounting example of a tube 1 according to the present embodiment.

As shown in FIG. 1, the tube 1 according to the present embodiment is mounted in a hole H that makes a body surface and the inside of lung parenchyma L2 of a lung L communicate with each other, and forms a port PT as a tube lumen. Specifically, the tube 1 is mounted in the hole H penetrating a thorax C1, a parietal pleura C2, and a visceral pleura L1 and the lung parenchyma L2 of the lung L, and forms the port PT for degassing to discharge the residual air to the outside of the body surface from a site where the residual volume is large (target site) in this lung parenchyma L2. This hole H is so formed as to avoid ribs CS located in the thorax C1. Although the hole H is formed at a site where the parietal pleura C2 and the visceral pleura L1 are fused to each other, the fusion does not need to be made.

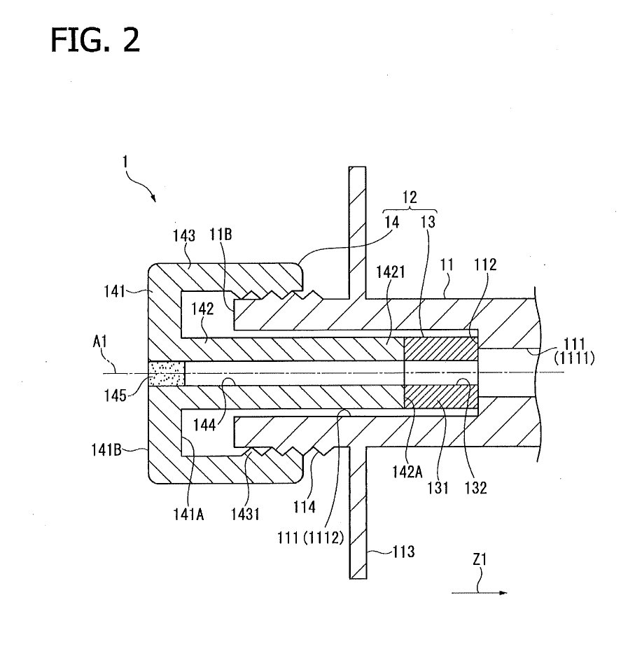

FIG. 2 is a partially enlarged sectional view of the tube 1.

As shown in FIG. 2, this tube 1 includes a main body portion 11 inserted into the hole H and a flow rate adjuster 12 provided for this main body portion 11.

The main body portion 11 is formed into a substantially cylindrical shape. An end surface 11A (see FIG. 1) thereof on the distal side in a Z1-direction, which is the direction of the insertion of the main body portion 11 into the hole H, is located in the lung parenchyma L2, and an end surface 11B on the proximal side in the Z1-direction is exposed to the outside of the body surface. In such a main body portion 11, a hole part 111 penetrating the main body portion 11 along the center axis A1 of this main body portion 11 is made. That is, the hole part 111 is opened to each of the end surface 11A and the end surface 11B.

Inside the main body portion 11 (i.e. inside the hole part 111), a step part 112 is formed at substantially the center in the Z1-direction in such a manner as to extend toward the radial outside of the hole part 111. This divides the hole part 111 into a first hole part 1111 that is located more distally in the Z1-direction than the step part 112 and has a smaller inner diameter and a second hole part 1112 that is located more proximally in the Z1-direction than the step part 112 and has a lager inner diameter. An elastic member 131 is disposed on this step part 112.

Furthermore, the main body portion 11 has a flange part 113 extending toward the radial outside of this main body portion 11. In other words, the flange part 113 extends along the body surface when the tube 1 is mounted in the hole H. An adhesive is provided at the site opposed to the body surface in this flange part 113. Due to this, the main body portion 11 is freely detachably set on the body surface.

Moreover, a helical thread groove 114 along the Z1-direction is formed in the outer surface of the end part of the main body portion 11 on the proximal side in the Z1-direction. To this thread groove 114, a thread 1431 formed in a pressure adjuster 14 to be described later is screwed.

The flow rate adjuster 12 is provided for the main body portion 11 and adjusts the discharge amount of the air flowing in the hole part 111 (residual air in a hollow L3). This flow rate adjuster 12 includes a diameter adjuster 13 and the pressure adjuster 14.

The diameter adjuster 13 has the elastic member 131 formed into an annular shape by rubber, synthetic resin, or the like having elasticity, and this elastic member 131 is so disposed that its end surface on the distal side in the Z1-direction abuts against the step part 112. At substantially the center of this elastic member 131, a penetration hole 132 that penetrates along the center axis of this elastic member 131 is formed. This center axis of the elastic member 131 substantially corresponds with the center axis A1 of the main body portion 11.

As described in detail later, the elastic member 131 is pressed toward the distal side in the Z1-direction by the pressure adjuster 14 to be compressed, and thereby the inner diameter of the penetration hole 132 becomes smaller. Due to this, the diameter of the flow path of the air in the hole part 111 is decreased, which adjusts the discharge amount of the air discharged from the lung parenchyma L2 (hollow L3) to the outside of the body surface via the tube 1.

The pressure adjuster 14 is attached to the end part of the main body portion 11 closer to the proximal side in the Z1-direction than the flange part 113 in such a manner as to be advanceable and retractable along the center axis A1 of this main body portion 11. The pressure adjuster 14 adjusts the pressure to the elastic member 131 forming the diameter adjuster 13 through advancement and retraction of this pressure adjuster 14.

Such a pressure adjuster 14 has a base part 141, an inserted part 142 and an outer shell part 143 that extend from this base part 141 toward the distal side in the Z1-direction, a hole part 144 penetrating the base part 141 and the inserted part 142 along the Z1-direction, and a filter 145.

The base part 141 is located on the proximal side of the main body portion 11 in the Z1-direction when the pressure adjuster 14 is attached to the main body portion 11. This base part 141 is formed into a substantially circular shape as viewed from the proximal side in the Z1-direction and the outer diameter of this base part 141 is larger than that of the main body portion 11.

The inserted part 142 is a cylindrical part inserted into the hole part 111. The outer diameter of this inserted part 142 is smaller than the inner diameter of the hole part 111. The distal part of this inserted part 142 in the Z-direction is a pressing part 1421 that presses the elastic member 131 disposed on the step part 112 toward the distal side in the Z1-direction. In association with the movement of the pressure adjuster 14 toward the distal side in the Z1-direction, the pressing part 1421 moves in the movement direction in the hole part 111 to press the elastic member 131 toward the distal side in the Z1-direction and elastically deform it.

The outer shell part 143 is a cylindrical part that is outside the inserted part 142 and surrounds an area of this inserted part 142 on the proximal side in the Z1-direction. The inner diameter of this outer shell part 143 is set to a size obtained by adding a predetermined clearance to the outer diameter of the main body portion 11, and the thread 1431 screwed to the thread groove 114 is formed in the inner surface of this outer shell part 143. Due to the screwing of these thread groove 114 and thread 1431, the pressure adjuster 14 advances and retracts relative to the main body portion 11 along the center axis A1 when being rotated around the center axis A1.

Between an end surface 141A of the base part 141 on the distal side in the Z1-direction and the end surface 11B of the main body portion 11 on the proximal side in the Z1-direction, a clearance to permit the maximum movement of the pressure adjuster 14 toward the distal side in the Z1-direction is set.

The hole part 144 is formed at the center of the pressure adjuster 14. One end thereof is opened to an end surface 142A of the inserted part 142 on the distal side in the Z1-direction and the other end is opened to an end surface 141B of the base part 141 on the proximal side in the Z1-direction. In this hole part 144, the air of the hollow L3 that has entered the hole part 111 flows.

The filter 145 is provided at a site in the hole part 144 on the proximal side in the Z1-direction. As such a filter 145, e.g. a hydrophobic filter can be employed. This can prevent entry of solids such as microorganisms, pathogens, and allergens into the lung parenchyma L2 and flow of liquids such as water into it.

### [Operation of Tube]

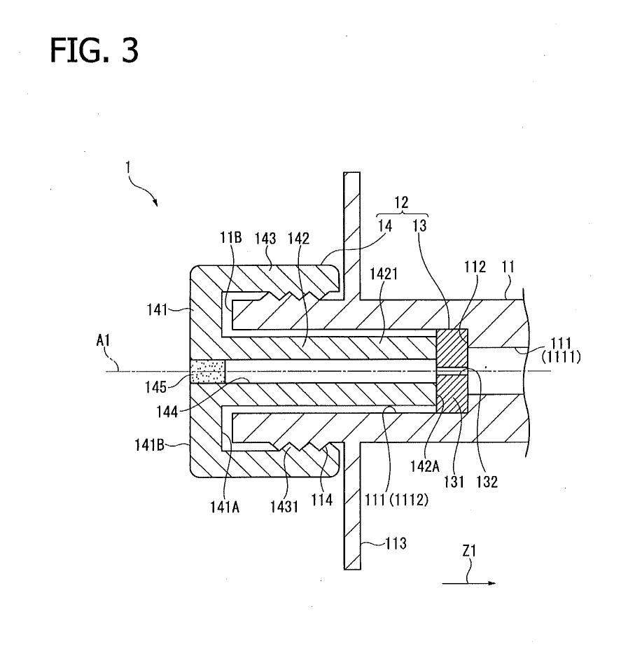

FIG. 3 is a sectional view of the tube 1 showing the state in which the elastic member 131 is compressed.

In the tube 1, when being rotated in one direction around the center axis A1, the pressure adjuster 14 moves relative to the main body portion 11 toward the distal side in the Z1-direction. Thereby, as shown in FIG. 3, the pressing part 1421 of the inserted part 142 presses the elastic member 131 provided on the step part 112.

The elastic member 131 to which the pressure is applied is elastically deformed. This decreases the inner diameter of the penetration hole 132. The penetration hole 132 makes the space in the hole part 111 in the main body portion 11 communicate with the space in the hole part 144. Therefore, due to the decrease in the inner diameter of the penetration hole 132, the diameter of the flow path of the air in the hollow L3 that is to enter the hole part 111 and be discharged from the hole part 144 to the outside of the body surface via the filter 145 is decreased, and thus the flow rate (i.e. discharge amount) of the air becomes lower. The inner diameter of such a penetration hole 132 changes depending on the pressure made to act on the elastic member 131 by the pressing part 1421. Therefore, the flow rate (i.e. discharge amount) of the air can be adjusted according to this pressure, i.e. the movement amount of the pressure adjuster 14.

The pressing part 1421 normally abuts against the end surface of the elastic member 131 on the proximal side in the Z1-direction to apply a predetermined pressure to this elastic member 131. Thus, the elastic member 131 is brought into tight contact with the step part 112 and the pressing part 1421.

Due to such a configuration, the air in the hollow L3 that has entered the hole part 111 is discharged to the outside of the body surface via the penetration hole 132 of the elastic member 131 and the hole part 144. Therefore, the following phenomenon is suppressed: the air flows along the outer surface of the inserted part 142 to flow between the thread groove 114 and the thread 1431 and be discharged to the outside of the body surface.

### Effects of First Embodiment

The following effects are provided by the tube 1 according to the present embodiment described above.

Because the flow rate adjuster 12 to adjust the flow rate of the air flowing in the hole part 111 is provided for the main body portion 11, the discharge amount of the air that flows in this hole part 111 and is discharged from the lung parenchyma L2 to the outside of the body surface can be adjusted. Therefore, the discharge amount of the air discharged from the lung parenchyma L2 to the outside of the body surface can be adjusted according to the residual functions of the lung, the individual difference in various kinds of lung functions, and the degree of progression of symptoms.

Furthermore, because the discharge amount of the air discharged from the lung L to the outside of the body surface can be adjusted, hyperdeflation of the lung L can be suppressed and therefore the occurrence of dyspnea can be suppressed.

The flow rate adjuster 12 includes the diameter adjuster 13 to adjust the diameter of the flow path of the air flowing in the hole part 111 according to the pressure adjusted by the pressure adjuster 14. According to this, the flow rate of the air can be decreased by decreasing the diameter of this flow path and the flow rate of the air can be increased by increasing the diameter of this flow path. Therefore, the discharge amount of the air discharged from the lung parenchyma L2 to the outside of the body surface can be surely adjusted.

The elastic member 131 disposed on the step part 112 in the hole part 111 is elastically deformed by being pressed by the pressing part 1421 located in the hole part 111 in the pressure adjuster 14, which advances and retracts along the center axis A1. This can adjust the inner diameter of the penetration hole 132 of the elastic member 131 based on the pressure that increases/decreases depending on the advancement/retraction amount of the pressing part 1421. Therefore, the diameter of the flow path of the air discharged from the lung parenchyma L2 to the outside of the body surface can be surely adjusted and hence the discharge amount of the air can be surely adjusted.

Due to the screwing of the thread groove 114 of the main body portion 11 and the thread 1431 of the pressure adjuster 14, the pressure adjuster 14 advances and retracts relative to the main body portion 11 along the Z1-direction by being rotated around the center axis A1. According to this, the pressure applied to the elastic member 131 by the pressing part 1421 can be kept with a simple configuration. This can suppress the return of the inner diameter of the penetration hole 132 adjusted through the elastic deformation of the elastic member 131 to the original diameter and can surely keep the adjusted discharge amount of the air.

Furthermore, because the pressing part 1421 can be made to advance and retract along the center axis A1 by rotating the pressure adjuster 14, the pressure applied to the elastic member 131 and the inner diameter of the penetration hole 132 can be easily adjusted and therefore the above-described discharge amount of the air can be easily adjusted.

### Second Embodiment

A second embodiment of the present invention will be described below.

A tube according to the present embodiment has a configuration similar to that of the above-described tube 1. The tube 1 has the configuration in which the thread groove 114 is formed in the outer surface of the main body portion 11 and the thread 1431 of the pressure adjuster 14 is screwed to the thread groove 114 outside the main body portion 11. In contrast, in the tube according to the present embodiment, the thread groove is formed in the inner surface of the main body portion and the thread of the pressure adjuster is screwed to the thread groove inside this main body portion. In this point, the tube according to the present embodiment is different from the above-described tube 1. In the following description, the same or substantially the same part as the part already described is given the same numeral and description thereof is omitted.

### [Configuration of Tube]

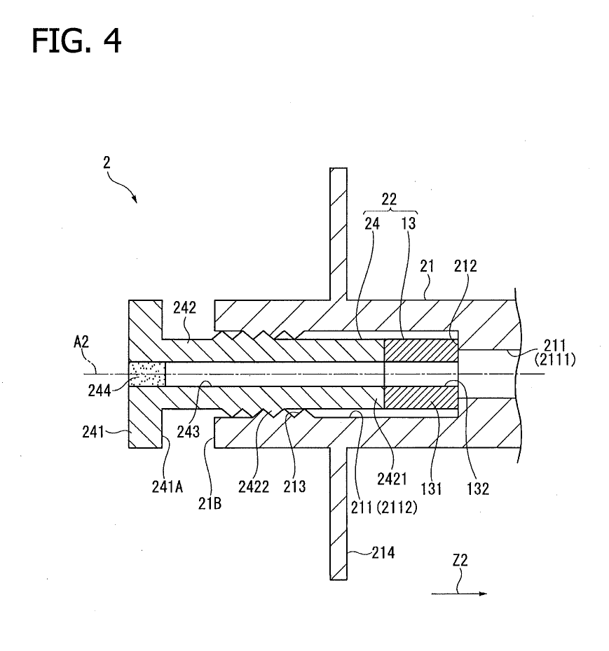

FIG. 4 is a sectional view showing a tube 2 according to the present embodiment.

Similarly to the above-described tube 1, the tube 2 according to the present embodiment is mounted in the hole H to make the outside of the body surface and the hollow L3 in the lung parenchyma L2 communicate with each other, and forms the port PT to discharge the residual air in this hollow L3 to the outside of the body surface. This tube 2 includes a main body portion 21 and a flow rate adjuster 22.

The main body portion 21 is formed into a substantially cylindrical shape. In this main body portion 21, a hole part 211 penetrating the main body portion 21 along a center axis A2 of the main body portion 21 is formed. One end of this hole part 211 is opened to the end surface (not shown) on the distal side in a Z2-direction, which is the direction of the insertion of the main body portion 21 into the hole H, and the other end is opened to an end surface 21B on the proximal side in this Z2-direction. The end surface on the distal side is exposed in the hollow L3 of the lung parenchyma L2.

Inside the main body portion 21, a step part 212 similar to the step part 112 is formed. By this step part 212, the hole part 211 is divided into a first hole part 2111 that is located more distally in the Z2-direction than this step part 212 and has a smaller inner diameter and a second hole part 2112 that is located more proximally in the Z2-direction than the step part 212 and has a larger inner diameter. Similarly to the above-described tube 1, the elastic member 131 of the diameter adjuster 13 is disposed on the step part 212 in such a manner that the surface perpendicular to the center axis in this elastic member 131 (i.e. surface on the distal side in the Z2-direction) abuts against this step part 212.

A helical thread groove 213 along the center axis A2 is formed on the proximal side in the Z2-direction in this second hole part 2112. To this thread groove 213, a thread 2422 of a pressure adjuster 24 to be described later is screwed.

Moreover, on the outer surface of the main body portion 21, a flange part 214 extending toward the radial outside of the main body portion 21 formed into a columnar shape is formed. Similarly to the above-described flange part 113, an adhesive is provided at the site opposed to the body surface in this flange part 214.

Similarly to the above-described flow rate adjuster 12, the flow rate adjuster 22 is provided for the main body portion 21 and adjusts the discharge amount of the air flowing in the hole part 211 (residual air in the hollow L3). This flow rate adjuster 22 includes the diameter adjuster 13 and the pressure adjuster 24.

The pressure adjuster 24 is a cylindrical member whose section along the Z2-direction is formed into a substantially T-shape. It advances and retracts along the center axis A2 and applies a pressure to the elastic member 131 of the diameter adjuster 13. This pressure adjuster 24 has a base part 241 having a substantially circular shape as viewed from the proximal side in the Z2-direction, an inserted part 242, and a hole part 243 penetrating the base part 241 and the inserted part 242 along the center axis A2. At a position in this hole part 243 on the proximal side in the Z2-direction, a filter 244 similar to the above-described filter 145 is provided.

The inserted part 242 is a cylindrical part extending from an end surface 241A of the base part 241 on the distal side in the Z2-direction toward the distal side in the Z2-direction. This inserted part 242 is inserted into the hole part 211 (specifically, second hole part 2112). The part of this inserted part 242 on the distal side in the Z2-direction is a pressing part 2421 that abuts against the elastic member 131 and presses this elastic member 131 in association with the movement of the pressure adjuster 24 toward the distal side in the Z2-direction. This pressing part 2421 applies a predetermined pressure to the elastic member 131 similarly to the above-described pressing part 1421.

On the outer surface of the inserted part 242 on the proximal side in the Z2-direction, the helical thread 2422 along the center axis of this inserted part 242 is formed. This thread 2422 is screwed to the thread groove 213 formed in the inner surface of the main body portion 21 when the inserted part 242 is inserted into the hole part 211. Due to this, the pressure adjuster 24 advances and retracts along the Z2-direction when being rotated around the center axis A2.

Between the end surface 241A of the base part 241 on the distal side in the Z2-direction and the end surface 21B of the main body portion 21 on the proximal side in the Z2-direction, a clearance to permit the maximum movement of the pressure adjuster 24 toward the distal side in the Z2-direction is set.

### [Operation of Tube]

In such a tube 2, similarly to the above-described tube 1, when the pressure adjuster 24 is rotated in one direction around the center axis A2, this pressure adjuster 24 moves relative to the main body portion 21 toward the distal side in the Z2-direction and the pressing part 2421 presses the elastic member 131. Thereby, the inner diameter of the penetration hole 132 formed in the elastic member 131 is adjusted and the diameter of the flow path of the air in the hollow L3 that is to enter the first hole part 2111 and be discharged to the outside of the body surface via this penetration hole 132 and the hole part 243 is adjusted. Therefore, the discharge amount of the air is adjusted.

### Effects of Second Embodiment

Such a tube 2 according to the present embodiment can provide effects similar to those of the above-described tube 1.

### Third Embodiment

A third embodiment of the present invention will be described below.

A tube according to the present embodiment is different from the above-described tubes 1 and 2 in that the configuration to adjust the diameter of the flow path of the air discharged from the inside of the hollow L3 to the outside of the body surface is different. In the following description, the same or substantially the same part as the part already described is given the same numeral and description thereof is omitted.

### [Configuration of Tube]

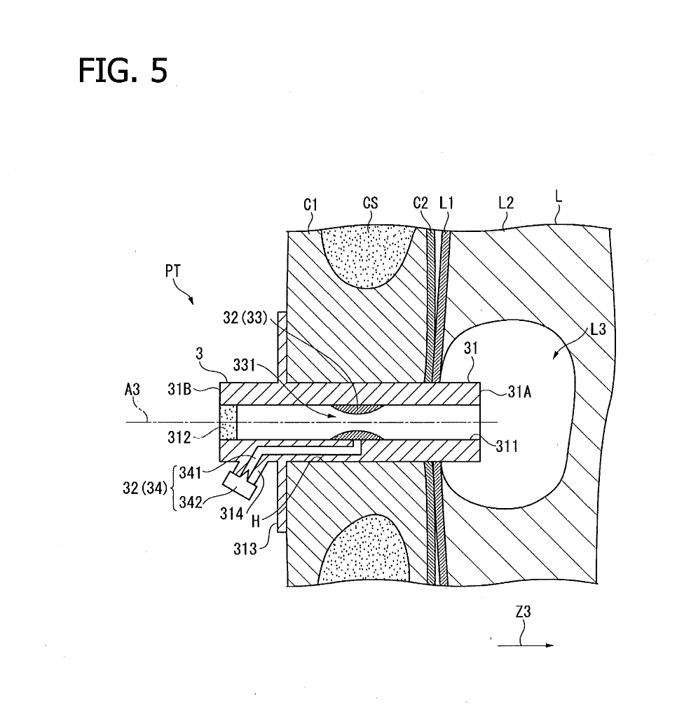

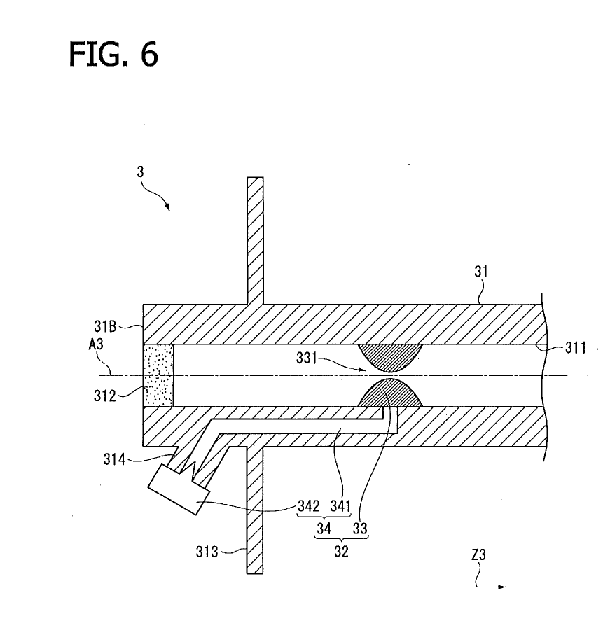

FIG. 5 is a sectional view showing a mounting example of a tube 3 according to the present embodiment. FIG. 6 is a sectional view showing this tube 3.

Similarly to the above-described tubes 1 and 2, the tube 3 according to the present embodiment is mounted in the hole H to make the outside of the body surface and the hollow L3 in the lung parenchyma L2 communicate with each other, and forms the port PT to discharge the residual air in this hollow L3 to the outside of the body surface. This tube 3 includes a main body portion 31 and a flow rate adjuster 32 as shown in FIGS. 5 and 6.

The main body portion 31 has a substantially cylindrical shape and is inserted into the hole H. In this main body portion 31, a hole part 311 penetrating the main body portion 31 along the center axis A3 of the main body portion 31 is formed. The distal side of the hole part 311 in a Z3-direction, which is the direction of the insertion of the main body portion 31 into the hole H, is opened to an end surface 31A (see FIG. 5) of this main body portion 31 on the distal side in the Z3-direction, and the proximal side in the Z3-direction is opened to an end surface 31B of the main body portion 31 on the proximal side in the Z3-direction. In the present embodiment, the inner diameter of the hole part 311 is constant.

In this hole part 311, a filter 312 similar to the above-described filter 145 is provided more proximally in the Z3-direction than a diameter adjuster 33 to be described later.

On the outer surface of the main body portion 31, a flange part 313 extending from this outer surface toward the radial outside of the main body portion 31 is provided. This flange part 313 is a part extending along the body surface when the main body portion 31 is mounted in the hole H, and the outer diameter of this flange part 313 is larger than the inner diameter of the hole H. An adhesive is provided at the site opposed to the body surface in this flange part 313. Due to this, the main body portion 31 is freely detachably set on the body surface.

At a position on the main body portion 31 located more proximally in the Z3-direction than the flange part 313, a tubular branch part 314 protruding from the outer surface of this main body portion 31 with an inclination from the center axis A3 toward the body surface is provided.

The flow rate adjuster 32 adjusts the flow rate of the air flowing in the above-described hole part 311. This flow rate adjuster 32 includes the diameter adjuster 33 and a pressure adjuster 34.

The pressure adjuster 34 injects a medium to the diameter adjuster 33 to thereby adjust the pressure to this diameter adjuster 33 and hence adjust the diameter of the flow path of the air in the hole part 311. This pressure adjuster 34 includes an injection path 341 and a check valve 342.

The injection path 341 is a channel for injecting the medium to the diameter adjuster 33 and is formed, in the main body portion 31, between the inner surface of the hole part 311 and the outer surface of this main body portion 31. One end of this injection path 341 is opened to substantially the center of the hole part 311 in the Z3-direction (i.e. site where the diameter adjuster 33 is disposed) and the other end is opened to the end surface of the branch part 314 on the tip side in the protrusion direction. In the present embodiment, a gas such as air is used as the medium. However, a liquid such as a normal saline solution may be utilized.

The check valve 342 is provided at the end part of the branch part 314 on the tip side in the protrusion direction from the outer surface of the main body portion 31. This check valve 342 prevents the medium injected into the injection path 341 opened to the end surface of the branch part 314 from flowing in reverse to leak out to the external.

The injection of the medium into the injection path 341, for which the check valve 342 is provided, is carried out by using a syringe or the like.

The diameter adjuster 33 is provided along the circumferential direction of the hole part 311 at the site where one end of the injection path 341 is opened in the hole part 311. That is, the diameter adjuster 33 is so provided as to make the circuit of the hole part 311 along the inner surface of this hole part 311.

### [Operation of Tube]

In the present embodiment, such a diameter adjuster 33 is formed of an annular body of rubber or the like (e.g. balloon) that expands or contracts depending on the amount of the above-described medium injected via the injection path 341. Furthermore, this diameter adjuster 33 has such a configuration that, due to the injection of the above-described medium, the diameter adjuster 33 changes from the state shown in FIG. 5 to the state shown in FIG. 6 and the inner diameter of a penetration hole 331 formed in the diameter adjuster 33 along the center axis A3 is reduced. Thus, according to the injection amount of this medium, the inner diameter of the penetration hole 331 and hence the diameter of the flow path of the air flowing in the hole part 311 can be adjusted. This can adjust the discharge amount of the air discharged from the hollow L3 to the outside of the body surface via the tube 3.

### Effects of Third Embodiment

The tube 3 according to the present embodiment described above can provide the following effects besides the same effects as those by the above-described tubes 1 and 2.

The diameter adjuster 33 expands or contracts according to the amount of medium supplied via the pressure adjuster 34 to thereby adjust the diameter of the flow path of the air flowing in the hole part 311. This can surely adjust the discharge amount of the air discharged from the lung parenchyma L2 to the outside of the body surface. Furthermore, because the diameter of the flow path is adjusted according to the amount of injected medium, adjustment of the discharge amount of the air is also easy.

The provision of the check valve 342 for the injection path 341 can suppress the occurrence of the situation in which the medium injected into this injection path 341 flows in reverse to leak out to the external and the adjusted diameter of the flow path of the air is changed. Therefore, the adjusted discharge amount of the air can be kept.

### Modifications of Embodiments

Although best configurations for carrying out the present invention are disclosed by the above description, the present invention is not limited thereto. Specifically, descriptions limiting shape, material, and so forth disclosed in the above are made in an exemplifying manner in order to facilitate understanding of the present invention and do not limit the present invention. Therefore, description with the name of a member from which part of the limitation of these shape, material, and so forth or all of the limitation is eliminated is encompassed in the present invention.

In the above-described first and second embodiments, the elastic member 131 of the diameter adjuster 13 pressed by the pressure adjuster 14 or 24 is elastically deformed. This moves the edge of the penetration hole 132 toward the radial inside or outside of this penetration hole 132. Thereby, the inner diameter of the penetration hole 132 is adjusted and hence the diameter of the flow path of the air discharged from the hollow L3 to the outside of the body surface is adjusted. In the third embodiment, the swell amount of the diameter adjuster 33 is adjusted according to the amount of medium injected from the injection path 341 and thereby the diameter of the flow path of the air is adjusted. However, the present invention is not limited thereto. That is, the discharge amount of the air may be adjusted by another method. For example, a configuration may be employed in which an object projects into or retracts from the hole part by driving by an actuator to decrease or increase the diameter of the above-described flow path.

In the above-described first and second embodiments, the elastic member 131 forming the diameter adjuster 13 is disposed on the step part 112 formed in the hole part 111. However, the present invention is not limited thereto. That is, the elastic member 131 may be disposed in any manner as long as pressure can be made to act on the elastic member 131 in association with advancement and retraction of the pressure adjuster 14.

In the above-described first and second embodiments, due to screwing of the threads 1431 and 2422 to the thread grooves 114 and 213, the pressure adjusters 14 and 24 are so attached to the main body portions 11 and 21 as to be advanceable and retractable along the center axes A1 and A2. However, the present invention is not limited thereto. Specifically, a configuration may be employed in which the pressure adjuster is so provided as to be advanceable and retractable relative to the main body portion based on a guide made along the center axis of the main body portion.

In the above-described third embodiment, the diameter adjuster 33 swells out according to the amount of medium injected via the injection path 341 to adjust the diameter of the above-described flow path. However, the present invention is not limited thereto. For example, a configuration may be employed in which a syringe or the like is inserted via an opening made in the side surface of the main body portion and the medium is directly injected into the diameter adjuster. Furthermore, in the hole part 311, the diameter adjuster 33 may be provided at a partial area along the circumferential direction of this hole part 311.

It should be understood by those skilled in the art that various modifications, combinations, sub-combinations and alternations may occur depending on design requirements and other factors insofar as they are within the scope of the appended claims or the equivalent thereof.

What is claimed is:

1. A tube mounted in a hole that makes a body surface and inside of lung parenchyma communicate with each other, the tube comprising:
a main body portion that has a hole part having one end opened to the inside of the lung parenchyma and the other end opened to outside of the body surface; and
a flow rate adjuster that is provided for the main body portion and adjusts a flow rate of air flowing in the hole part.

2. The tube according to claim 1, wherein
the flow rate adjuster includes
a diameter adjuster that is provided in the hole part and adjusts a diameter of a flow path of the air flowing in the hole part according to an applied pressure, and
a pressure adjuster that adjusts the pressure applied to the diameter adjuster.

3. The tube according to claim 2, wherein
the main body portion has a step part that is formed in the hole part and extends toward radial outside of the hole part,
the diameter adjuster is disposed on the step part and has an elastic member having an annular shape,
the elastic member has a penetration hole penetrating the elastic member,
an inner diameter of the penetration hole is adjusted according to the applied pressure, and
the pressure adjuster has a pressing part that advances and retracts in the hole part and applies a pressure to the elastic member.

4. The tube according to claim 3, wherein
the main body portion has a helical thread groove that is formed in one of an outer surface of the main body portion and an inner surface of the hole part and is along a center axis of the main body portion, and
the pressure adjuster has a thread screwed to the thread groove and is so configured as to be advanceable and retractable along the center axis of the main body portion.

5. The tube according to claim 2, wherein
the diameter adjuster is provided in the hole part and expands toward radial inside of the hole part according to pressure of a supplied medium to adjust the diameter of the flow path, and
the pressure adjuster adjusts an amount of medium supplied to the diameter adjuster.

6. The tube according to claim 5, wherein
the pressure adjuster includes
an injection path that is formed in the main body portion and injects the medium to the diameter adjuster, and
a check valve that is provided on an opposite side to a side of the diameter adjuster in the injection path and prevents reverse flow of the injected medium.
